(19) United States
(12) Patent Application Publication
(10) Pub. No.: US 2026/0338755 A1
(43) Pub. Date: Oct. 29, 2026

(54) SYSTEMS AND METHODS FOR ASSESSING EXPOSURE TO PESTICIDES

(71) Applicant: The University of North Carolina at Charlotte, Charlotte, NC (US)

(72) Inventors: Jun Wang, Charlotte, NC (US); Xiuxia Du, Charlotte, NC (US); Hussian Maanaki, Charlotte, NC (US)

(21) Appl. No.: 19/188,804

(22) Filed: Apr. 24, 2025

(51) Int. Cl. G01N 33/18; G01N 27/07

(52) U.S. Cl. CPC G01N 33/184; G01N 27/07

(57) ABSTRACT

Rapid and accurate detection of personal exposure to or contamination with OP pesticides is critical to both research and clinical care for victims. However, this detection remains highly variable across commercially available assays, e.g., the Ellman method, and such assays may also not adequately detect low but clinically relevant levels of exposure biomarkers. Moreover, these assays are generally performed in the centralized laboratories, and is slow and expensive. These deficits translate into missed opportunities for the timely initiation of the most appropriate treatment and interventions for acutely or chronically poisoned victims. A simple, rapid, non-or minimally invasive biomonitoring tool for onsite sensitive and accurate detection of personal exposure to or contamination with OP pesticides is provided.

### CROSS REFERENCE TO RELATED APPLICATIONS

This application is a continuation-in-part and claims priority and the benefit of, co-pending U.S. Non-Provisional application Ser. No. 18/086,961, filed on Dec. 22, 2022, which claims priority to U.S. provisional application no. 63/306337, filed on Feb. 3, 2022, the entire contents of which are hereby incorporated by reference.

### GOVERNMENT SUPPORT CLAUSE

This invention was made with government support under R41 ES032388 awarded by the National Institute of Health (NIH). The government has certain rights in the invention.

### FIELD

The disclosed technology relates to systems and methods for assessing exposure to pesticides. Specifically, this disclosed technology relates to a nanosensor coupled to a processing device for detecting the presence of organophosphate pesticides based on analysis of a blood, food, or water sample.

### BACKGROUND

Organophosphate (OP) pesticides constitute a large class of chemical insecticides that are widely used in the agricultural industry and at home across the nation and around the world. More than 200 OP pesticides have been formulated into thousands of different products, which accounts for about 40% of total pesticide products. Exposure to OP pesticides remains a major environmental and occupational health risk factor because these pesticides are well-established neurotoxicants based upon their capability to inhibit acetylcholinesterase (AChE) in the nervous system. People at elevated risk include workers in agriculture, manufacturing industries, greenhouses, or florists, occupants of residences and public places, including schools, in close proximity to the OP application sites, and agriculture worker's family members via carry-over exposure. Each year, more than 2 million migrant and seasonal farmworkers work in the United States. It was found that these farmworkers and their family members are widely exposed to OP pesticides at work and at home. This exposure results in decreased cholinesterase activity.

There are two primary types of OP poisoning: acute and chronic. Acute OP poisoning (roughly >50% blood cholinesterase (ChE) inhibition) can evoke an accumulation of the neurotransmitter acetylcholine (ACh) in the synaptic cleft, leading to cholinergic crisis with paralysis of muscles and ultimately to death. Chronic OP poisoning is more common (<20% blood ChE inhibition, acute cholinergic signs are not elicited but oxidative stress, genotoxicity, and other effects may occur) and could contribute to various diseases including Parkinson's disease, Alzheimer's disease, reproductive disorders, and cancers. In view of these findings, timely and accurate detection of environmental and occupational exposure to OP pesticides is critical for ensuring safety.

Biomonitoring is an effective approach for timely and accurate detection of exposure to OP pesticides since it can quantitatively associate an internal dose with a measurable biological effect. It has also been exhibited by epidemiology studies that accurate assessment of chemical exposures along with biological effects will have the most meaningful interpretation and thus have maximal impact. However, a major impediment in conducting quantitative biomonitoring for exposure assessment is the lack of rapid, cost-effective, field deployable, quantitative technologies that measure chemical exposure/response biomarkers.

Further, the monitoring of pesticide residue in food and water is important for public health. Chromatography coupled with mass spectroscopy (MS) is a standard method for detecting pesticides, such as through the use of gas chromatography (GC) and high-performance liquid chromatography (HPLC) with MS. However, disadvantages to these techniques include complex sample preparation protocols, high operation and maintenance costs, time-consuming methods, and centralized laboratories with trained personnel requirements. These disadvantages make such conventional methods of detection generally unsuitable for on-site pesticide detection.

For blood samples, both blood AChE and butyrylcholinesterase (BChE) inhibitions by OP pesticides are important biological markers for the indication of OP exposure and poisoning because OP pesticides can inhibit both red blood cell (RBC) AChE and plasma BChE in blood and RBC AChE inhibition is correlated to the nervous-system AChE inhibition. Thus, blood ChE activity assay is used to measure ChE inhibition in clinical laboratories for detecting OP poisoning. The most widely used ChE activity assay method is the Ellman assay, a photometric method. In addition, a number of other methods have been developed including, but not limited to, a fluorescence assay, an electrochemical (EC) assay, a Michel (ΔpH) assay, a pH-stat assay, the Walter Reed Army Institute of Research Whole Blood (WRAIR WB) assay, and a radiochemical assay. However, most of these methods are not field-deployable and have to be performed in laboratories.

In addition, these conventional methods of measuring ChE inhibition suffer from confounding problems when detecting and assessing OP exposure: (i) conventional methods are less accurate and may provide false-negative and false-positive results because of interference from sulfhydryl groups (R-SH) in samples and the light absorption from sample matrix (e.g., Hemoglobin); (ii) a long turnaround time requiring at least two clinical visits to measure the pre-exposure blood ChE baseline and post-exposure blood ChE in a clinical laboratory; and (iii) a high expense because of the required complex instruments, trained technicians, and labor.

All these limitations call for a rapid, cost-effective, sensitive POC biomonitoring device for onsite detection of OP exposure. At present, some commercially available in-field tests exist, but suffer from several disadvantages including interference from R-SH and cost. Additionally, conventional tools for measuring OP exposure face difficulty in seamlessly processing, analyzing, reporting, storing, and sharing data generated during experimentation. To address these challenges and limitations, a rapid, cost-effective, sensitive POC biomonitoring tool for onsite detection of OP exposure is highly demanded.

Embodiments of the present disclosure are directed to these and other considerations.

### SUMMARY

Organophosphate (OP) pesticides poisoning is a major environmental and occupational health problem worldwide due to its acute or chronic toxicity. Rapid and accurate detection of personal exposure to OP pesticides is critical to both research and clinical care for victims. However, this detection remains highly variable across commercially available assays, e.g., the Ellman method, and such assays may also not adequately detect low but clinically relevant levels of exposure biomarkers. Moreover, these assays are generally performed in the centralized laboratories, and is slow and expensive. These deficits translate into missed opportunities for the timely initiation of the most appropriate treatment and interventions for acutely or chronically poisoned victims. A simple, rapid, non or minimally invasive biomonitoring tool for onsite sensitive and accurate detection of personal exposure to OP pesticides is highly desirable to address the drawbacks of conventional solutions. Aspects of the present disclosure are directed to a nanosensor system for onsite rapid and sensitive detection of OP exposure using a tiny drop of finger-stick blood. According to some embodiments, the nanosensor system takes advantage of nanotechnology and wireless technology and utilize an important biomarker, blood cholinesterase, for the detection of personal OP exposure and the resulted tool has the potential to serve as an in-field diagnostic tool for environmental and occupational workers. The disclosed nanosensor platform is a simple, rapid, cost-effective, and minimally-invasive, point-of-care biomonitoring tool for accurate personal exposure assessment and for on-site rapid detection of exposure to OP pesticides. Thus, the disclosed nanosensor platform can serve as a personal meter for self-test just like a glucose meter and can also be used in field, physician's office, bedside, and emergency room, among other use cases.

The disclosed nanosensor platform is additionally configured to detect pesticide analytes, including OP pesticides, in food and water samples. Food samples include unprocessed and processed food items for human consumption. Water samples include drinking water and environmental water samples, where environmental water includes well water, river water, and water from other bodies of water and moving water sources. For non-liquid samples, including food samples, the sample may be centrifuged with water, shaken, re-centrifuged, and have supernatant utilized as the sample for analysis.

Disclosed embodiments may include a nanosensor configured to detect pesticide analytes in a sample. The nanosensor may include an interdigitated electrode (IDE) and a nanomaterial deposited on the IDE. The nanomaterial can include a carbon nanoparticle phase and a polyaniline phase. The sample may be a blood sample, a food sample, or a water sample, including an environmental water sample. The blood sample may be from a subject with exposure or suspected exposure to pesticides. The pesticide analytes are organophosphate (OP) pesticides in some instances.

Disclosed embodiments may include a system for detecting pesticide analytes in a sample. The system can include a nanosensor configured to receive the sample. The system can include a measurement component operatively coupled to the nanosensor and configured to generate data associated with a measured conductivity change of the nanosensor in response to an addition of the sample to the nanosensor. The system can include a processing device operatively coupled to the measurement component. The processing device can include one or more processors and a non-transitory memory in communication with the one or more processors and storing instructions thereon that when executed by the one or more processors are configured to cause the processing device to perform the steps of a method. The method may include receiving, from the measurement component, the data associated with the measured conductivity change and detecting the presence of pesticide analytes based on the data.

Disclosed embodiments may include a method. The method may include applying a sample to a nanosensor. The method may include generating, by a measurement component operatively coupled to the nanosensor, data associated with a measured conductivity change of the nanosensor in response to applying the sample to the nanosensor. The method can include receiving, by a processing device operatively coupled to the measurement component, the data associated with the measured conductivity change. The method may include determining, by the processing device, whether the sample includes pesticide analytes based on the data.

### BRIEF DESCRIPTION OF THE DRAWINGS

Reference will now be made to the accompanying drawings, which are not necessarily drawn to scale, and which illustrate various implementations, aspects, and principles of the disclosed technology. In the drawings:

FIG. 1 is a block diagram of an example nanosensor system that may be used to assess exposure of a subject to pesticides, according to exemplary embodiments consistent with the present disclosure.

FIG. 2 is a block diagram of an example processing device that may be used to assess exposure of a subject to pesticides, according to exemplary embodiments consistent with the present disclosure.

FIG. 3A depicts a photo of an exemplary nanosensor platform and processing device, according to exemplary embodiments consistent with the present disclosure. [A] denotes the nanosensor. On top of the sensing area is a glass fiber ring pad preloaded with AChE inhibitor and/or anti-buffer agents and a glass fiber circle preloaded with substrates; [B] denotes a digital multimeter measurement component for measuring resistance and transmitting the data to a smartphone via Bluetooth; [C] denotes a smartphone processing device that receives measurement data from the multimeter.

FIG. 3B is a transmission electron microscopy (TEM) image of the nanostructure of the nanosensor of FIG. 3A, according to exemplary embodiments consistent with the present disclosure.

FIG. 3C depicts normalized response curves of nanosensors, according to exemplary embodiments consistent with the present disclosure. Calibration curve of the nanosensor for measuring AChE in blood samples under optimal conditions. Heat-denatured whole blood spiked with different concentrations of human AChE (U/mL): (a) 0.0, (b) 2.0, (c) 5.0, (d) 10.0, (e) 15.0, (f) 18.0, and (g) 20.0. Data is normalized for varying cholinesterase levels, with a linear calibration curve provided to demonstrate the relationship between cholinesterase levels and nanosensor response generation.

FIG. 3D depicts normalized responses curves of nanosensors, according to exemplary embodiments consistent with the present disclosure. Calibration curve of the nanosensor for measuring BChE in whole blood under optimal conditions. Heat-denatured whole blood spiked with different concentrations of BChE (U/mL): (a) 0.0, (b) 0.5, (c) 1.0, (d) 1.5, (e) 2.0, (f) 3.0, and (g) 5.0. Data is normalized for varying cholinesterase levels, with a linear calibration curve provided to demonstrate the relationship between cholinesterase levels and nanosensor response generation.

FIG. 3E is a graphical representation of responses of nanosensors to blood samples with and without the anti-buffer treatment, according to exemplary embodiments consistent with the present disclosure.

FIG. 3F is a graphical representation of responses of nanosensors to blood samples in vitro spiked with different concentrations of an organophosphate contaminant, according to exemplary embodiments consistent with the present disclosure.

FIG. 3G is a graphical representation of responses of nanosensors to different volumes of finger-stick blood from a donor, according to exemplary embodiments consistent with the present disclosure. Volumes of whole blood are as indicated: (a) 1.0 μL. (b) 1.5 μL. (c) 2.0 μL.

FIG. 4A is a graphical representation of a normalized response curve and corresponding enzyme activity from a measured blood sample, according to exemplary embodiments consistent with the present disclosure.

FIG. 4B is a graphical representation of calculated enzyme inhibition for a control sample and a sample including organophosphate pesticides, according to exemplary embodiments consistent with the present disclosure.

FIG. 5A is a schematic illustration of a protocol to fabricate a nanosensor. The Au IDE is cleaned with acetone and water, respectively.

FIG. 5B is a schematic illustration of a protocol to fabricate a nanosensor. A sensing area is confined using a template.

FIG. 5C is a schematic illustration of a protocol to fabricate a nanosensor. A PAnNW/CNT suspension is applied on the Tween-20-pretreated IDE surface and allowed to dry.

FIG. 5D is a schematic illustration of a protocol to fabricate a nanosensor. A protective coating is applied on the surface of PAnNW/CNT nanomaterial coating to reduce the blood matrix effect.

FIG. 5E is a schematic illustration of a protocol to fabricate a nanosensor. The template is removed.

FIG. 5F is a photo depicting five nanosensors fabricated with a protocol consistent with the present disclosure.

FIG. 6 is a schematic illustration of an exemplary CS/MWCNT/PAnNF smartphone-nanosensor for the detection of human exposure to OP pesticides using AChE as a model in accordance with the present disclosure. Finger-stick whole blood sample is applied to an AuIDE coated with the CS/MWCNT/PAnNF/nanocomposite. A whole blood sample containing AChE and BChE interacts with the nanosensor that is pre-treated with a glass fiber outer pre-treatment pad. After pre-treatment (ChE-specific), an ACh substrate loaded glass fiber pad is placed onto the sensing surface and rapidly dissolves. Prior to OP pesticide exposure, AChE is fully active and rapidly hydrolyzes dissolved ACh to produce a large quantity of protons, which heavily dopes the CS/MWCNT/PAnNF film. However, upon exposure to OP pesticides, AChE activity is depreciated, which reduces the amount of protons generated, resulting in a slightly-doped CS/MWCNT/PAnNF film. The CS/MWCNT/PAnNFs-coated AuIDE is connected to a multimeter which measures resistance change overtime and transmits resistive data to a smartphone through Bluetooth. Smartphone app processes resistive data and displays changes in AChE/BChE activity for pre-and post-exposure. Pesticide-induced inhibition of AChE and/or BChE results in reduced proton generation, thus a reduced resistance change.

FIG. 7A depicts a photograph of an exemplary nanosensor platform with a pre-treatment pad applied, according to exemplary embodiments consistent with the present disclosure.

FIG. 7B depicts a photograph of an exemplary nanosensor platform with a pre-treatment pad and a sample applied, according to exemplary embodiments consistent with the present disclosure.

FIG. 7C depicts a photograph of an exemplary nanosensor platform after sample application and placement of the substrate pad, according to exemplary embodiments consistent with the present disclosure.

FIG. 7D depicts a photograph of an exemplary measurement component and nanosensor platform according to exemplary embodiments consistent with the present disclosure.

FIG. 7E depicts an exemplary display of a processing device according to exemplary embodiments consistent with the present disclosure.

FIG. 8A is a graphical representation of the inhibition of AChE in 2 μL whole blood by different concentrations of BW284c51 (μM): (a) 0.0, (b) 1.0, (c) 5.0, (d) 10.0, (e) 25.0, (f) 75.0, (g) 150.0, and (h) 300.0. An incubation period of 5 minutes and 3 mM ACh was used.

FIG. 8B is a graphical representation of optimization of pretreatment time for whole blood with (solid line) 0.2 mM MgCl₂+CaCl₂ and (dashed line) 150 μM BW284c51 inhibitor and 0.2 mM MgCl₂+CaCl₂ at periods of 0.0, 1.0, 2.0, 3.0, 4.0, and 5.0 minutes with 3 mM ACh.

FIG. 9 is a graphical representation of long-term stability (0, 2, 4, 12, 28, 33, and 42 weeks) of MWCNT/PAnNF/CS-AuIDE nanosensors for AChE measurements in whole blood. MWCNT/PAnNF/CS-AuIDE nanosensors were stored under constant dry vacuum conditions.

FIG. 10A is a graphical comparison of AChE measurements from the nanosensor-smartphone platform and radiometric method using four whole blood samples spiked with 5 different concentrations of Diazinon-Oxon. AChE inhibition by Diazinon-Oxon is shown.

FIG. 10B is a graphical comparison of AChE inhibition measurements from the nanosensor-smartphone platform and radiometric method using four whole blood samples spiked with 5 different concentrations of Paraoxon-methyl. AChE inhibition by Paraoxon-methyl is shown.

FIG. 10C is a graphical comparison of BChE inhibition measurements from the nanosensor-smartphone platform and radiometric method using four whole blood samples spiked with 5 different concentrations of Diazinon-Oxon. BChE inhibition by Diazinon-Oxon is shown.

FIG. 10D is a graphical comparison of BChE inhibition measurements from the nanosensor-smartphone platform and radiometric method using four whole blood samples spiked with 5 different concentrations of Paraoxon-methyl. BChE inhibition by Paraoxon-methyl is shown.

FIG. 10E is a graphical comparison of AChE inhibition measurements from the nanosensor-smartphone platform and radiometric method using four whole blood samples spiked with 5 different concentrations of Diazinon-Oxon or Paraoxon-methyl. Linear model for AChE inhibition between nanosensor and radiometric method.

FIG. 10F is a graphical comparison of AChE inhibition measurements from the nanosensor-smartphone platform and radiometric method using four whole blood samples spiked with 5 different concentrations of Diazinon-Oxon or Paraoxon-methyl. Bland Altman plot for AChE inhibition between the nanosensor and radiometric method.

FIG. 10G is a graphical comparison of BChE inhibition measurements from the nanosensor-smartphone platform and radiometric method using four whole blood samples spiked with 5 different concentrations of Diazinon-Oxon or Paraoxon-methyl. Linear model for BChE inhibition between the nanosensor and radiometric method.

FIG. 10H is a graphical comparison of BChE inhibition measurements from the nanosensor-smartphone platform and radiometric method using four whole blood samples spiked with 5 different concentrations of Diazinon-Oxon or Paraoxon-methyl. Bland Altman plot for BChE inhibition between the nanosensor and radiometric method.

FIG. 10I is a graphical comparison of AChE activity measurements from the nanosensor-smartphone platform and Ellman's method using a subset of four whole blood samples spiked with 5 different concentrations of Diazinon-Oxon or Paraoxon-methyl. Linear model for AChE activity between the nanosensor and Ellman's method with 28 samples.

FIG. 10J is a graphical comparison of AChE activity measurements from the nanosensor-smartphone platform and radiometric method using a subset of four whole blood samples spiked with 5 different concentrations of Diazinon-Oxon or Paraoxon-methyl. Bland Altman plot for AChE activity between the nanosensor and Ellman's method.

FIG. 11A depicts a graphical representation of AChE inhibition an exemplary nanosensor and by radiometric analysis methods for 22 samples, according to exemplary embodiments consistent with the present disclosure. Samples were from farmworkers during field testing and are finger-stick whole blood samples. Half of the collected samples were analyzed via radiometric method and the other half using the exemplary nanosensor with a 2 μL volume and 10 minute testing time.

FIG. 11B depicts a graphical representation of BChE inhibition an exemplary nanosensor and by radiometric analysis methods for 22 samples, according to exemplary embodiments consistent with the present disclosure. Samples were from farmworkers during field testing and are finger-stick whole blood samples. Half of the collected samples were analyzed via radiometric method and the other half using the exemplary nanosensor with a 2 μL volume and 10 minute testing time.

FIG. 11C depicts a graphical comparison of nanosensor and radiometric analysis of AChE inhibition of FIG. 11A according to exemplary embodiments consistent with the present disclosure.

FIG. 11D depicts a graphical comparison of nanosensor and radiometric analysis of BChE inhibition of FIG. 11B according to exemplary embodiments consistent with the present disclosure.

FIG. 12 is a flow diagram illustrating an exemplary method for constructing a nanosensor, according to exemplary embodiments consistent with the present disclosure.

FIG. 13 is a flow diagram illustrating an exemplary method for assessing the exposure of a subject to a pesticide based on a blood sample, according to exemplary embodiments consistent with the present disclosure.

Further implementations, features, and aspects of the disclosed technology, and the advantages offered thereby, are described in greater detail hereinafter, and can be understood with reference to the following detailed description, accompanying drawings, and claims.

### DETAILED DESCRIPTION

Some implementations of the disclosed technology will be described more fully with reference to the accompanying drawings. This disclosed technology may, however, be embodied in many different forms and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed devices, systems, and methods.

Reference will now be made in detail to example embodiments of the disclosed technology that are illustrated in the accompanying drawings and disclosed herein. Wherever convenient, the same reference numbers will be used throughout the drawings to refer to the same or like parts.

Embodiments of the present disclosure are directed to a nanosensor system for on-site, rapid, and sensitive measurement of blood ChE activity and detection of exposure to OP pesticides using a finger-prick blood sample. In some embodiments, the nanosensor may be of a nanosensor, a measurement component, and a processing device. In some embodiments, the nanosensor can be constructed of a nanomaterial-modified interdigitated electrode (IDE). In some embodiments, the measurement component may be a digital multimeter with wireless connectivity (e.g., via WiFi™, Bluetooth™, etc.). In some embodiments, the processing device can be a personal computing device, such as a smartphone, tablet, laptop, or desktop with integrated application. In some embodiments, the nanosensor can include nanomaterial comprising carbon nanotube/polyaniline nanowires (CNT/PAnNWs), which may be deposited on an IDE serving as a transducer for the nanosensor. According to some embodiments, the operative principle of the nanosensor is based on the hydrolysis of acetylcholine (ACh) by AChE or BCh by BChE to generate protons doping polyaniline nanowires, which leads to a change of transport properties of CNT/PAnNWs. Therefore, the quantification of ChE activity in samples can be easily achieved by monitoring the conductance changes of CNT/PAnNWs using the measurement component. The sensing signals will be wirelessly transmitted (e.g., via Bluetooth™) to the processing device, which processes the raw data and displays the results. Besides processing, analyzing, and reporting data, the processing device can also store data locally and share data with professionals such as healthcare providers, epidemiologists, etc. by an internet connection to a cloud platform.

Embodiments consistent with the present disclosure are directed to a nanosensor system for onsite rapid and sensitive detection of OP exposure using finger-stick blood samples. In some embodiments, the nanosensor is reagentless (self-contained reagents) and the sensor strip of the nanosensor may be single use and disposable just like a glucose sensor strip. Thus, such a platform is a real sample-to-answer device, straightforward, easy to use, and user-friendly. This nanosensor system provides a new avenue for simple, rapid, inexpensive self-testing and field POC detection of OP exposure in environmental and occupational settings. The tool can be readily utilized for military applications against chemical warfare agents, homeland countermeasures against OP nerve agents, and efficacy testing of therapeutically anticholinergic drugs for Alzheimer's disease.

Embodiments consistent with the present disclosure are directed to a nanosensor system for the rapid and sensitive detection of pesticide analytes from food or water samples. In these samples, the food or water source may be contaminated with pesticides, including OP pesticides. As described for blood samples, the quantification of ChE activity in samples can be achieved by monitoring the conductance changes of CNT/PAnNWs using the measurement component. Thus, the sample-to-answer device can be utilized to detect the presence of pesticide analytes as contaminants or toxic compounds in food and water for human consumption, and in environmental water sources.

Nanomaterials, including nanoparticles, nanotubes, nanowires, etc., can greatly enhance the sensitivity of given assays. Conducting polymer nanowires (CPNWs) have been used for chemo-/biosensors because of their high surface areas, chemical specificities, tunable conductivities, material flexibilities, and easy processing at low cost. But until now, there has been no utilization of the unique conductivity property of CPNWs and its hybrid with CNTs for measuring blood ChE activity and detecting OP exposure using blood samples. In previous works, polyanline (PAn) has been used to immobilize enzyme ChE on electrodes and to electrocatalytically oxidize enzymatic product thiocholine for the EC detection of OP pesticides, where the same enzyme substrate acetylthiocholine is used as the Ellman method. Some PAn/CNT/ChE modified electrodes have been developed for EC detection of OP pesticides in water, taking advantage of PAn and CNT's electrocatalytic oxidation of thiocholine. In contrast to the nanofibers used within embodiments of the present disclosure, the previously electropolymerized PAn films on electrodes were generally bulk materials. Previously deployed EC methods also suffer from the same interference of R-SH in a sample as the Ellman method. Moreover, for these EC methods, ChE is immobilized on an electrode through PAn to measure its activity loss due to the enzyme inhibition by OP pesticides in a sample. In contrast, the disclosed embodiments takes advantage of the unique and sensitive conductance response of PAnNWs to enzymatic product protons using substrate acetylcholine for resistive measurement of blood ChE activity in a sample. Thus, the disclosed approach is different from these EC methods in terms of the fabrication method, detection principle, technique, and the analytical target. Some of the disclosed embodiments use PAn nanowires rather than PAn bulk materials and avoid both the light absorption and R-SH interferences from a sample that is typical in other detection approaches. As a result, the nanosensor systems consistent with the disclosed embodiments are more sensitive and accurate than the Ellman method, EC methods, and other conventional methods described above.

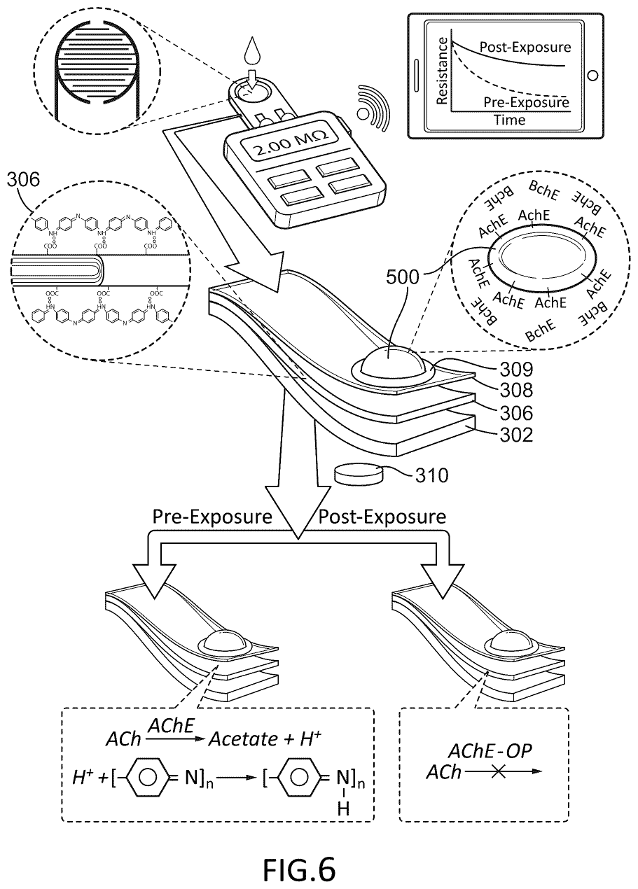

Aspects consistent with the present disclosure provide many advantages, including but not limited to the following: (1) Integrating a nanosensor with a processing device to achieve a sample-to answer biomonitoring device for onsite assessing OP exposure and poisoning, Through the processing device, the nanosensor system can seamlessly process, analyze, display, store, and share data and achieve a new mobile health in environmental and occupational settings, making public health care better and more efficient. Embodiments of the disclosed technology also offers an opportunity to provide healthcare remotely. (2) Embodiments of the present disclosure combine the unique biologically catalytic properties of ChE with the unique conductance property of PAnNWs for resistive measurement of blood ChE activity, which make the blood ChE test more sensitive, convenient, and at a lower cost than with conventional methods. (3) Using a minimal finger-stick blood sample and the integration of all sensing components (e.g., sensing film, enzyme substrate, enzyme inhibitor, and anti-inference reagents as described in more detail below with respect to FIG. 6) into a single platform to achieve a rapid, portable, and easy to use biomonitoring device for onsite detection of exposure to OP pesticides.

The disclosed methods, devices, and systems results in a novel POC biomonitoring tool for assessing OP poisoning, which is able to provide a result quickly (e.g., run in approximately 10 minutes), inexpensively (e.g., approximately $2 per sample tasted), and with a low detection limit (e.g., approximately 0.11 U/mL AChE and 0.093 U/mL BChE). The disclosed methods, devices, and systems, are accurate, provide for reproducible results, come in a portable package, and operates in a minimally invasive manner capable of performing analysis with a minimal blood sample (e.g., a volume of less than approximately 2 μL).

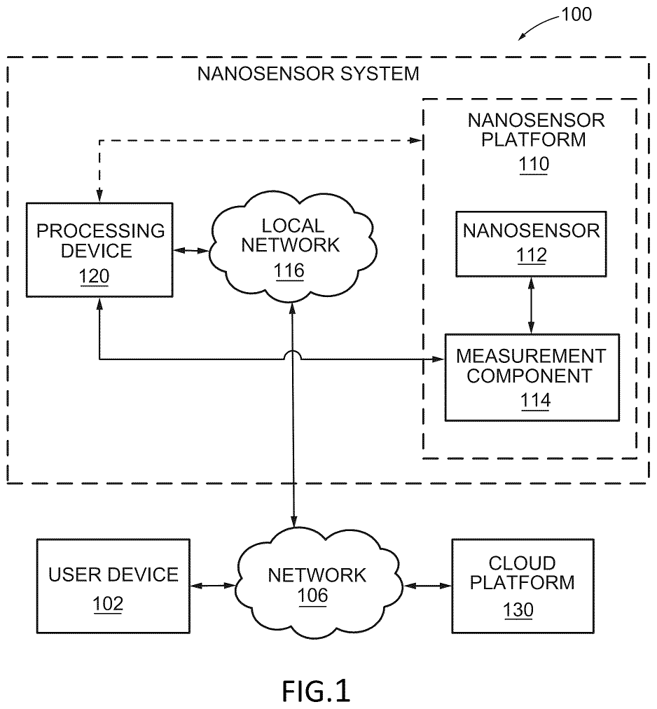

FIG. 1 is a block diagram of an example nanosensor system 100 in which processing device 120 may operate, according to an example implementation of the disclosed technology. The components and arrangements shown in FIG. 1 are not intended to limit the disclosed embodiments as the components used to implement the disclosed processes and features may vary. As shown, processing device 120 may interact with components of nanosensor platform 110 and with a local network 116. The local network 116 interacts and with a cloud platform 130 via network 106. In certain example implementations, the nanosensor platform 110 can include a nanosensor 112 and a measurement component 114, although in other embodiments, nanosensor 112 and measurement component 114 can be integrated into a single device. In certain example implementations, processing processing device 120, nanosensor 112, and measurement component 114 of nanosensor system 100 can be integrated into a single device. In certain example implementations, the system can include a local network 116 and a cloud platform 130.

In some embodiments, a user may operate the processing device 120 and/or user device 102. The processing device 120 and/or user device 102 can include one or more of a mobile device, smart phone, general purpose computer, tablet computer, laptop computer, telephone, public switched telephone network (PSTN) landline, smart wearable device, voice command device, other mobile computing device, or any other device capable of communicating with the local network 116 and/or network 106 and ultimately communicating with one or more components of the nanosensor system 100 and/or cloud platform 130. In some embodiments, the user device 102 and/or the processing device 120 may include or incorporate electronic communication devices for hearing or vision impaired users.

According to some embodiments, the processing device 120 may include an environmental sensor for obtaining audio or visual data, such as a microphone and/or digital camera, a geographic location sensor for determining the location of the device, an input/output device such as a transceiver for sending and receiving data, a display for displaying digital images, one or more processors, and a memory in communication with the one or more processors.

The processing device 120 may include programs (scripts, functions, algorithms) to configure data for visualizations and provide visualizations of datasets and data models on the processing device 120. This may include programs to generate graphs and display graphs. The processing device 120 may include programs to generate histograms, scatter plots, time series, or the like on the processing device 120. The processing device 120 may also be configured to display properties of data models and data model training results including, for example, architecture, loss functions, cross entropy, activation function values, embedding layer structure and/or outputs, convolution results, node outputs, or the like on the processing device 120.

The network 106/116 may be of any suitable type, including individual connections via the internet such as cellular or WiFi networks. In some embodiments, the network 106/116 may connect terminals, services, and mobile devices using direct connections such as radio-frequency identification (RFID), near-field communication (NFC), Bluetooth™, low-energy Bluetooth™ (BLE), WiFi™, ZigBee™, ambient backscatter communications (ABC) protocols, USB, WAN, or LAN. Because the information transmitted may be personal or confidential, security concerns may dictate one or more of these types of connections be encrypted or otherwise secured. In some embodiments, however, the information being transmitted may be less personal, and therefore the network connections may be selected for convenience over security.

The network 106/116 may include any type of computer networking arrangement used to exchange data. For example, the network 106/116 may be the Internet, a private data network, virtual private network (VPN) using a public network, and/or other suitable connection(s) that enable(s) components in the nanosensor system 100 environment to send and receive information between the components of the system 100. The network 106 may also include a PSTN and/or a wireless network.

Cloud platform 130 may include a computer system configured to store data and results generated by nanosensor system 100. Cloud platform 130 may include a computer system configured to receive communications from processing device 120 via for example, a mobile application, a chat program, an instant messaging program, a voice-to-text program, an SMS message, email, or any other type or format of written or electronic communication. Cloud platform 130 may have one or more processors and one or more databases, which may be any suitable repository of website data. Information stored in cloud platform 130 may be accessed (e.g., retrieved, updated, and added to) via local network 116 and/or network 106 by one or more devices or systems of system 100. In some embodiments, cloud platform 130 may host websites or applications that may be accessed by the user device 102. Cloud platform 130 may also be hosted by an online provider of website hosting, networking, cloud, or backup services, such as Microsoft Azure™ or Amazon Web Services™.

In accordance with certain example implementations of the disclosed technology, the cloud platform 130 may include one or more computer systems configured to compile data from a nanosensor system 100. The processing device 120 can correlate compiled data, analyze the compiled data, arrange the compiled data, generate derived data based on the compiled data, and store the compiled and derived data in a database such as the cloud platform 130. The cloud platform may serve as a back-up storage device and may contain data and information that is also stored on, for example, processing device 120.

In some embodiments, nanosensor 112 can be implemented as an interdigitated electrode that is modified with certain nanomaterials, such as multi-wall carbon nanotubes and/or polyaniline nanowires. According to some embodiments, a user can add a small (e.g., 2 μL) blood sample to be tested for the presence of an organophosphate pesticide to the nanosensor 112. The nanosensor 112 can undergo a change in conductance in response to the addition of the blood sample, the results of which are recorded by an interconnected measurement component 114. According to some embodiments, the measurement component 114 can be implemented as a multimeter tool that is configured to record the conductance change caused by the addition of a blood sample to the nanosensor 112. In some embodiments, measurement component 114 is configured with a form of wireless communication, such as RFID, NFC, Bluetooth™, low-energy Bluetooth™, WiFi™, ZigBee™, ABC, USB, WAN, and/or LAN, that allows measurement component 114 to communicate with other components within nanosensor system 100, such as with processing device 120.

Processing device 120 is configured to receive the data measured by measurement component 114 (e.g., changes in conductance of nanosensor 112) and process the data in order to determine whether a subject has been exposed to organophosphate pesticides based on the blood sample. While the detection of organophosphates is one example of what can be detected based on a change in conductance of nanosensor 112, embodiments consistent with the present disclosure are not so limited. In other embodiments, the change in conductance of nanosensor 112 can be used to as an early detection method for various diseases that can affect a subject's ability to break down choline (e.g., by inhibiting cholinesterase), such as but not limited to Parkinson's disease, Alzheimer's disease, and other neurodegenerative disorders, sudden infant death syndrome (SIDS), chronic liver disease, reproductive disorders, and various cancers.

According to some embodiments, the results generated by the nanosensor system 100 can be transmitted and stored by cloud platform 130. Cloud platform 130 can be utilized to allow third parties, such as healthcare providers, epidemiologists, etc. to access and assess the results generated by nanosensor system 100.

User device 102 can be used by a third party, such as a healthcare provider, epidemiologist, etc. to access results stored on cloud platform 130 for analysis. According to some embodiments, the data stored in cloud platform 130 can be permissioned such that the third party (e.g. user of user device 102) may need to provide appropriate authentication (e.g., a username, a password, etc.) in order to access data stored in cloud platform 130. In yet other embodiments, a third party (e.g., via user device 102) can directly access data generated by nanosensor system 100 (e.g., via one or more of network 106 and network 116).

Although the preceding description describes various functions of user device 102, nanosensor 112, measurement device 114, processing device 120, and/or cloud platform 130 in some embodiments, some or all of these functions may be carried out by a single computing device.

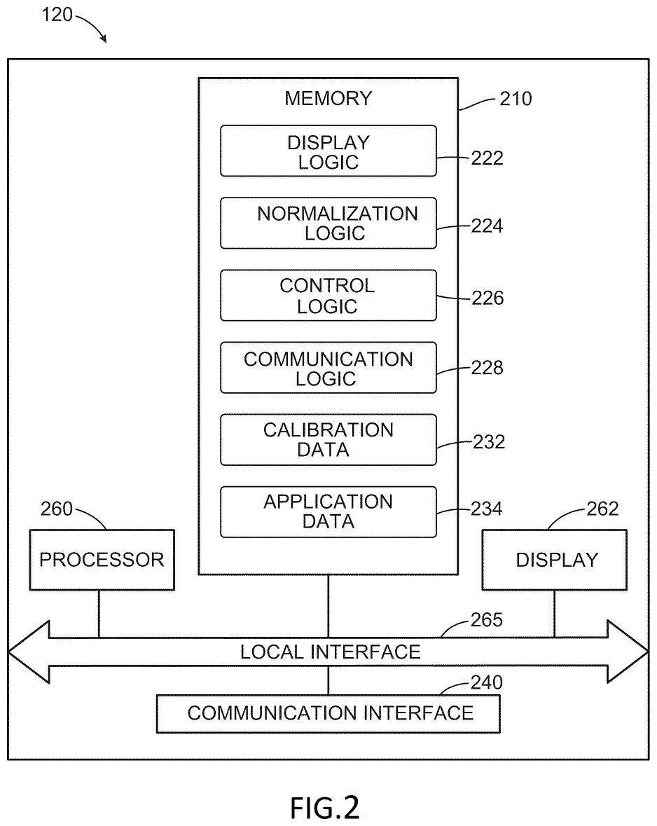

FIG. 2 depicts an example schematic diagram of certain components of processing device 120 in accordance with some embodiments of the present disclosure. The processing device 120 may include a memory 210. As used herein, the processing device 120 may include a memory 210. As used herein, memory 210 may refer to any suitable storage medium, either volatile and non-volatile (e.g., RAM, ROM, EPROM, EEPROM, SRAM, flash memory, disks or optical storage, magnetic storage, or any other tangible or non-transitory medium) that stores information that is accessible by a processor. Memory 210 may store instructions used in the systems and methods described herein. While FIG. 2 illustrates a single discrete memory 210, it will be understood that the embodiments described herein are not limited to any particular arrangement and that other embodiments may store information in one combined memory, in one or more memories, some local to the other components illustrated in FIG. 2 and/or some shared with, or geographically located near, other remote computing systems.

The illustrated embodiment depicts a number of modules stored in memory 210, specifically display logic 222, normalization logic 224, control logic 226, and communication logic 228. Memory 210 can also include data stores such as calibration data 232 and application data 234. These depicted modules may variously represent one or more algorithms, computational models, decision making rules or instructions, or the like implemented as software code or computer-executable instructions (i.e., routines, programs, objects, components, data structures, etc.) that, when executed by one or more processors 260, program the processor(s) to perform the particular functions of their respective logic. These modules are depicted in FIG. 2 as several discrete components, each labelled as an individual “logic”, however, in various embodiments, the functions of each respective logic may be executable on their own or as part of one or more other modules; that is, any configuration of the depicted logical components may be used, whether implemented by hardware, software, firmware, or any combination thereof. The capabilities of these various logics are described in greater detail below.

The processing device 120 may include control logic 226, including one or more algorithms or models for generally controlling the operation of the processing device 120. The memory 210 may also, in one embodiment, include communication logic 228, including one or more algorithms or models for obtaining information from or communicating information via network 106 and/or network 116 (FIG. 1). The processing device 120 may, via communication interface 240, operate to exchange data with various components and/or devices on the network 106, network 116, or any other network. For instance, communication interface 240 and communication logic 228 may be used to access data from nanosensor platform 110 (e.g., in the manner(s) described in greater detail below). In some embodiments, communication logic 228 may use APIs provided by one or more of cloud platform 130 and nanosensor system 100 to obtain or transmit stored data, however, other methods of data collection and transmission may alternatively be used such as one or more software development kits, which may include, e.g., one or more application programming interfaces (APIs), web APIs, tools to communicate with embedded systems, or any other appropriate implementation.

While communication logic 228 is illustrated as being a separate logical component, in an alternative embodiment, the processing device 120 may include communication logic 228 as part display logic 222, normalization logic 224, and/or control logic 226. In another alternative embodiment, the communication logic 228 may communicate with the control logic 226 to read or write data to memory 210 or to another data repository (not shown) within the processing device 120.

In some embodiments, processing device 120 may be implemented in whole or in part as a machine learning system (e.g., neural network software) for achieving the functionalities described herein. In one embodiment, one or more of display logic 222 and normalization logic 224, control logic 226, and/or communication logic 228 (or any subset of any of those logics) may be implemented at least in part as one or more machine learning algorithms.

While, in the exemplary embodiment, each of display logic 222, normalization logic 224, control logic 226, and communication logic 228 is depicted as part of processing device 120, these logical components need not be so configured, and in other embodiments, other configurations of the various components, within content generation system 120 or distributed over one or more computing systems, are possible. display logic 222, normalization logic 224, control logic 226, and communication logic 228 may be variously implemented in software, hardware, firmware or any combination thereof. In the exemplary processing device 120 shown in FIG. 2 display logic 222, normalization logic 224, control logic 226, and communication logic 228 are implemented in software and are stored in memory 210 of the processing device 120. Note that these components, when implemented in software, can be stored and transported on any non-transitory computer-readable medium for use by or in connection with an apparatus (e.g., a microprocessor) that can execute instructions. In the context of this disclosure, a “computer-readable medium” can be any device or system that can contain or store a computer program for use by or in connection with an instruction execution apparatus.

The logics of the exemplary processing device 120 depicted in FIG. 2 may be executed by one or more conventional processing elements 260, such as one or more central processing units (CPU), digital signal processors (DSP), graphics processing units (GPU), application-specific integrated circuits (ASICs), field-programmable gate arrays (FPGAs), and/or microprocessors programmed with software or firmware, other specialized processor or combination of processors, or other circuitry that communicates to and drives the other elements within the processing device 120 via a local interface 265, which can include at least one bus, such as I2C, SPI, USB, UART, and GPIO. As an example, the processor 260 may execute instructions of software stored in memory 210, such as display logic 222, normalization logic 224, control logic 226, and communication logic 228, or subsets thereof. While FIG. 2 illustrates one processor 260 which implements all of the various logics in the processing device 120, it is possible in other embodiments for the processing device 120 to employ multiple processors. In one such alternate embodiment, discrete processing elements may be used for each of (or any subset of display logic 222, normalization logic 224, control logic 226, and communication logic 228, or any portions or subsets of those logics. In some embodiments, the processing of processing device 120 is not limited to being performed by a processing element connected to the local interface 265, but instead, any portion of processing in support of the various logics of processing device 120 may be distributed over one or more computer systems that may be remotely located. For instance, processing device 120 may include physical computing devices residing at a particular location or may be deployed, wholly or partially, in a cloud computing network environment (e.g., cloud platform 130). In this description, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.). In some embodiments, the processor 260 may comprise an artificial neural network or other type of configuration for performing machine learning functions based on instructions stored in memory 210.

Memory 210 may be configured, in some embodiments, to include information stored as calibration data 232 and application data 234. In other embodiments, any or all of these components and/or information need not be stored on memory 210, but may instead be stored in a different memory and database, whether local processing device 120 or on one or more remote servers (in some embodiments, in the cloud).

Application data 234 includes programming and instructions for enabling data visualization, data processing, analysis, and commenting of data generated by nanosensor system 100. Normalization logic 224 is configured to use calibration data 232 to normalize the raw data collected by nanosensor platform 110. Once raw data is normalized according to normalization logic 224, the normalized data can be used to determine a change in conductance in the nanosensor 112 which can be correlated to a change in cholinesterase activity of a blood sample applied to the nanosensor 112. Calibration data 232 can include the conductance of nanosensor 112 when a blood sample is applied that is known to be devoid of cholinesterase inhibitors. In this way, normalization logic 224 can use calibration data 232 to accurately calculate cholinesterase inhibition present within a test blood sample.

In some embodiments, display logic 222, control logic 226 and/or communication logic 228 are executed by processor 260 to provide a graphical user interface to be displayed on the processing device 120, cloud platform 130, and/or user device 102. For example, display logic 222, control logic 226 and/or communication logic 228 may present to the user device 102, a user interface through which a user can analyze the data generated by nanosensor system 100. Similarly, display logic 222, control logic 226 and/or communication logic 228 can provide a graphical user interface for a user of processing device 120 (e.g., the subject or a healthcare provider assisting a subject) to view and analyze the data generated by nanosensor system 100.

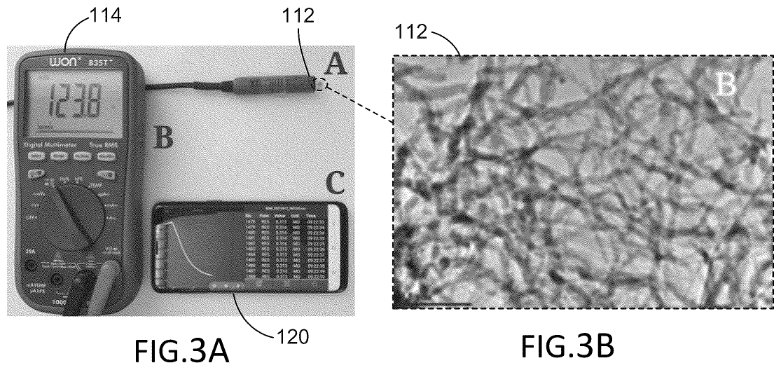

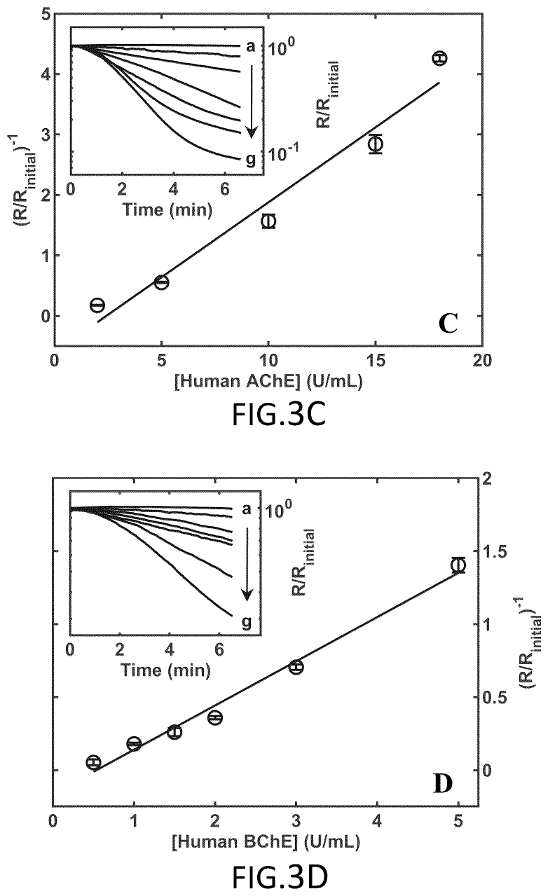

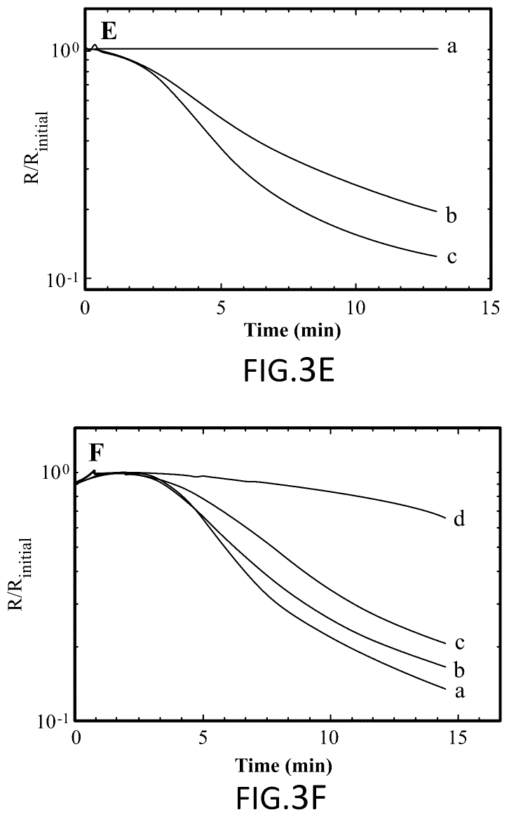

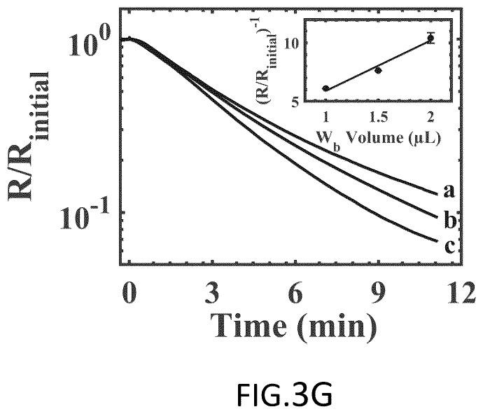

Experimental results of disclosed embodiments are summarized below. FIG. 3A displays a photo of the nansensor system 100 in which a CNT/PAnNW-coated IDE nanosensor (A) is connected to a digital meter (B) with Bluetooth, which can transmit data to a smartphone with an APP (C). While not visible in FIG. 3A, a pre-treatment pad 309 and a substrate pad (e.g., substrate 310) pre-loaded with ACh are located on the top of the sensing area of an IDE sensor. FIG. 3B shows a transmission electron microscopy (TEM) image of CNT/PAnNWs that were synthesized in lab according to methods known in the art. It can be seen from FIG. 3B that the CNT/PAnNWs are uniform with diameters of approximately 40 to 60 nm.

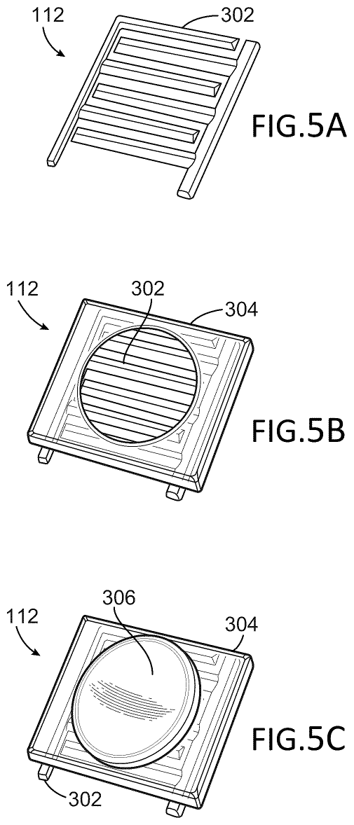

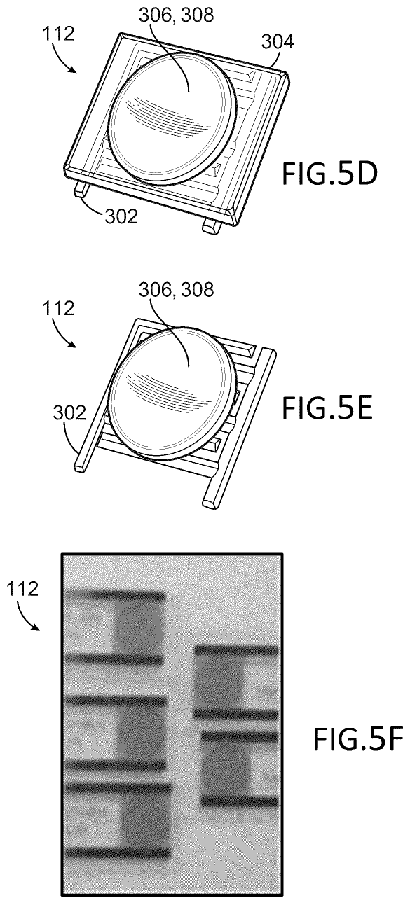

Investigators established a protocol (FIGS. 5A-5F) for fabricating the nanosensors and found that devices fabricated with this protocol showed good uniformity and good reproducibility for measuring blood enzyme activity with the RSD of about 2% (20 devices). Devices were fabricated using this protocol for further studies.

The responses of CNT/PAnNW-coated nanosensors versus AChE and BChE activity shown in FIGS. 3C and 3D. The graphs represents normalized responses of nanosensors of the present disclosure to six replicates of a blank sample (control, heat denatured blood, no ChE) and each concentration of AChE or BChE indicated. Human AChE (U/mL) in FIG. 3C is as indicated: (a) 0.0, (b) 2.0, (c) 5.0, (d) 10.0, (e) 15.0, (f) 18.0, and (g) 20.0. BChE (U/mL) in FIG. 3D is as indicated: (a) 0.0, (b) 0.5, (c) 1.0, (d) 1.5, (e) 2.0, (f) 3.0, and (g) 5.0. According to FIG. 3C the calibration curve equation for AChE is y=0.24786x−0.59987 with the R² value at 0.98, detection limit at 0.11 U/mL, and a dynamic range between 2.0 U/mL and 18.0 U/mL. The calibration curve equation for BChE (FIG. 3D) is y=0.30262×−0.16209 with the R² value at 0.99, detection limit at 0.093 U/mL, and a dynamic range between 0.5 and 5.0 U/mL. The nanosensor is highly reproducible with CV for AChE and BChE calibration curve data at 2.07 % and 1.15%, respectively. These calibration curves were integrated into the mobile app for measuring AChE and BChE activity in whole blood samples. The total analysis time for each measurement, including the pre-treatment stage, is approximately 8 min.

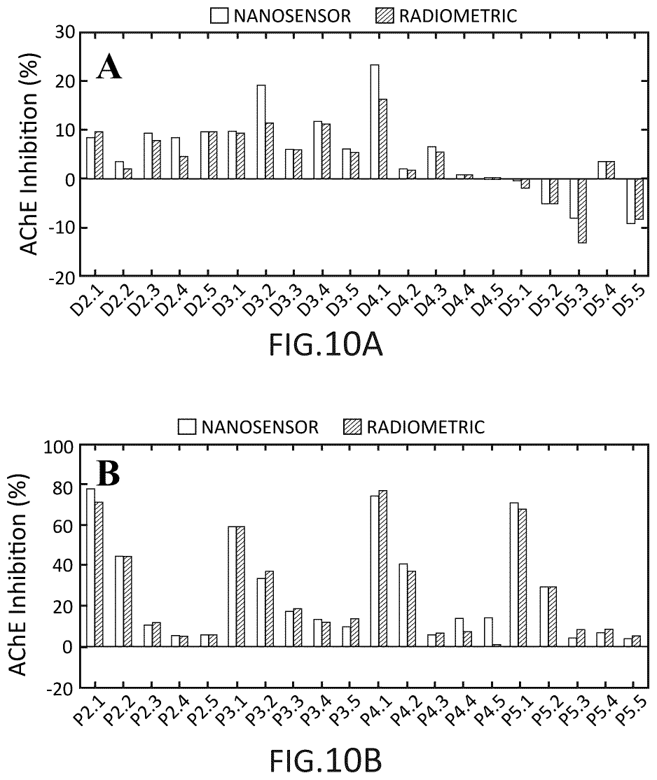

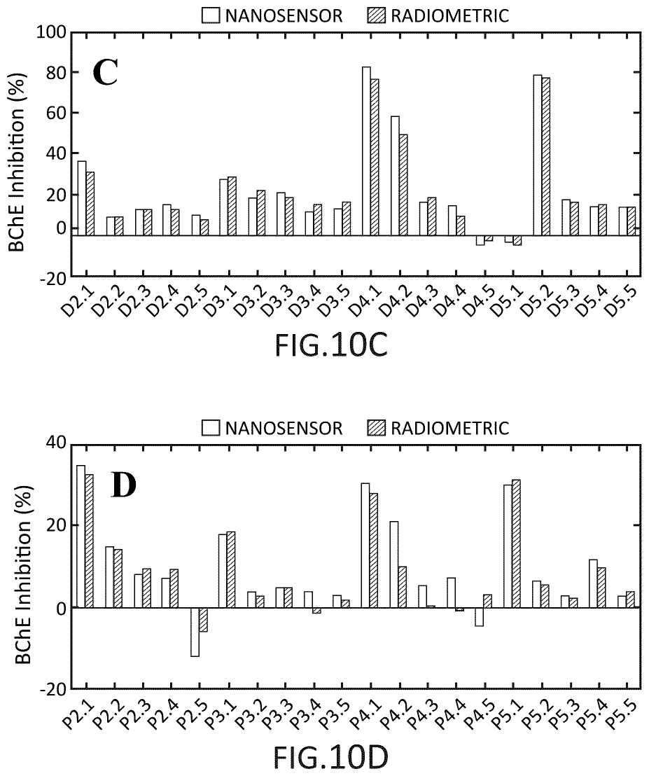

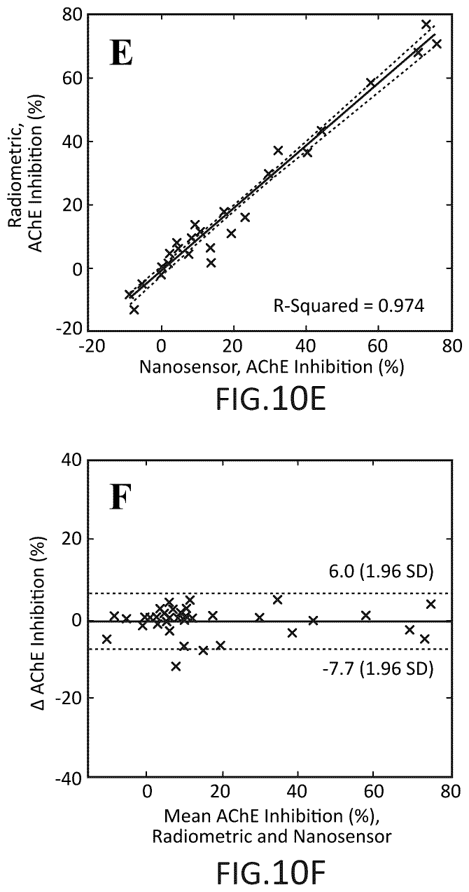

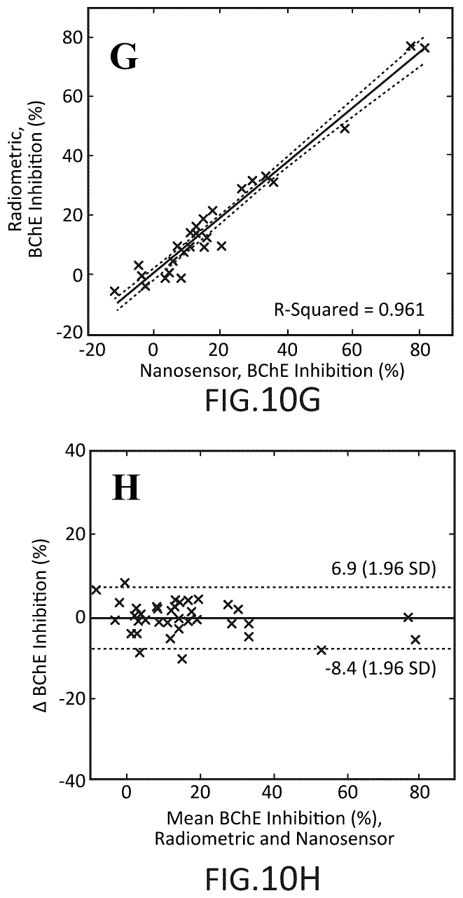

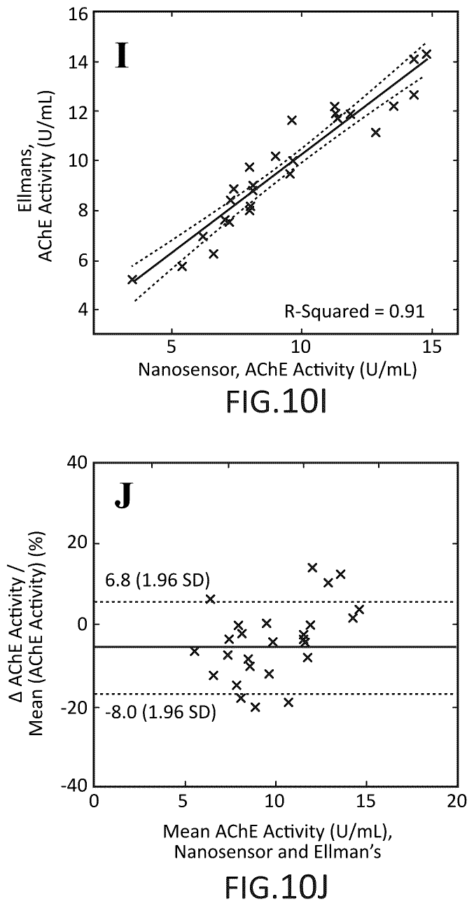

In order to test the possibility of the nanosensor system 100 for measuring enzyme activity in blood, the effect of blood buffer system (normally around pH7.4) on the sensor was explored (FIG. 3E). In FIGS. 3E, 10 mM Mg²⁺ and Ca²⁺ were utilized for blood sample treatments where indicated: (a) A control. 1.5 μL of blood treated with 10 mM Mg²⁺ and Ca²⁺ but no ACh; (b) 1.5 μL of blood without treatment with Mg²⁺ and Ca²⁺ and with ACh; (c) 1.5 μL of blood treated with 10 mM Mg²⁺ and Ca²⁺ and with ACh. When there was no treatment of a blood with Ca²⁺ and Mg²⁺, the nanosensor showed a low response (FIG. 3E (b)) because some of the protons generated by enzymatic reaction on the surface of the sensor were neutralized by the blood buffer system. However, after treating a blood sample with cations Ca²⁺ and Mg²⁺ to reprecipitate CO₃²⁻ and PO₄³⁻ in blood, it was found the nanosensor exhibited a larger response (FIG. 3E(c)). If there was no ACh (a control), the sensor showed no response to blood treated even with the cations Ca²⁺ and Mg²⁺ (FIG. 3E (a)). The results indicate that the use of cations Ca²⁺ and Mg²⁺ to treat the blood can destroy its buffer capacity and minimize the effect of blood buffers on the nanosensor. Moreover, the nanosensor was used to test blood samples in vitro dosed with different concentrations of paraoxon (FIG. 3F). In FIG. 3F, paraoxon concentrations of (a-d, ppb) 0, 0.48, 4.8, 48 with ACh on a pad were examined. All blood samples were treated with Mg²⁺ and Ca²⁺. It can be seen in FIG. 3F that the blood enzyme activity was deceased with the increase in paraoxon (FIG. 3F (a-d)). Finally, this nanosensor was used to test finger-stick blood samples with different volumes from 1 μL to 2 μL (see FIG. 3G). In particular, finger-stick blood sample volumes of (a-c, μL) 1.0, 1.5, 2.0 were tested. The inset is a plot of responses vs. blood volumes. The blood was treated with Mg²⁺ and Ca²⁺. The results show that the responses of nanosensors increase linearly with the increase in blood volume under the tested conditions.

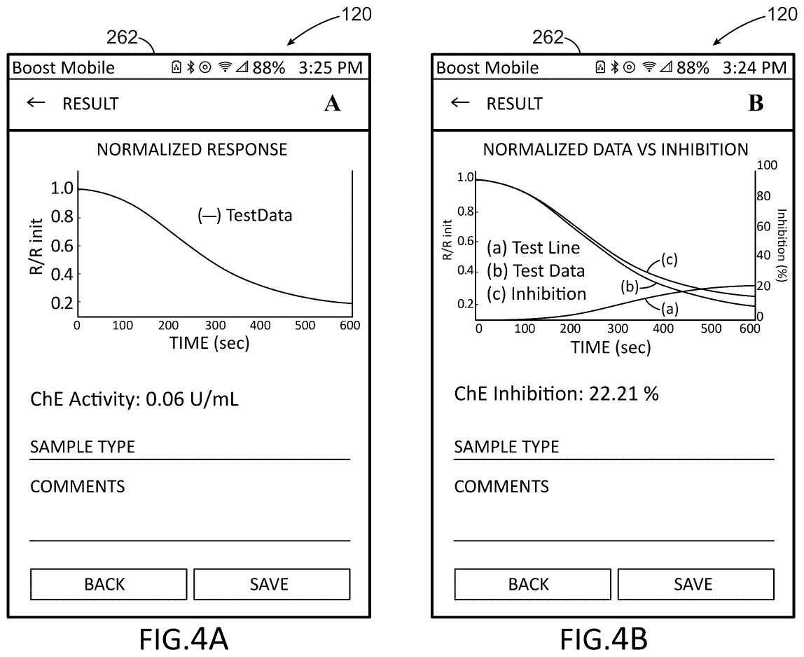

The processing device 120 is equipped with multiple functions including wireless data transmission, data processing and analysis (e.g. normalization, alignment, peak recognition, enzyme activity determination through a pre-defined and built-in calibration curve, enzyme inhibition calculations), data visualization and user interface, data storage, and comments. FIGS. 4A-4B shows some functions of the processing device 120 for processing and analyzing data. In FIGS. 4A-4B, an AChE sample without OP pesticides is shown (b), as well as one spiked with OP pesticides (c) and the enzyme inhibition curve (a). As can be seen from FIG. 4A, the processing device 120 can normalize raw data and display a normalized curve for a measurement and automatically calculate the enzyme concentration and display the result on the screen. Moreover, the processing device 120 can align normalized curves and automatically calculate the inhibition from two measurements and directly display enzyme inhibition results on the screen (see FIG. 4B (a)). According to a facile chemical synthesis method, CNT/PAnNW with different amount of multi-wall CNT (e.g., 0, 0.5, 1.0, 1.5 wt %) have been synthesized and characterized by TEM. According to some embodiments, CNT/PAnNW with CNT 1.0 wt % showed overall best performance in terms of the sensitivity of response to protons, easiness of fabricating a uniform film, and stability, but it should be understood that in other embodiments a different amount of multi-wall CNT can be included within the nanosensor 112. FIGS. 5A-5F shows the protocol for fabricating a nanosensor 112. In FIG. 5A, the IDE 302 is cleaned, while in FIG. 5B a sensing area is confined using a template 304 with a hole in the diameter of approximately 3 mm attached on the IDE 302, though other hole diameters are possible. As shown in FIG. 5C, a nanomaterial coating 306 (e.g., a PAnNW/CNT suspension) is applied on the IDE 302 surface and allowed to dry. According to some embodiments, the surface of IDE 302 may be pretreated with Tween-20 prior to application of nanomaterial coating 306. According to some embodiments and as shown in FIG. 5D, a protective coating 308 is dropped on the surface of nanomaterial coating 306 and allowed to dry. The protective coating 308 protects the nanomaterial coating 306 and reduces the blood matrix effect. According to some embodiments, the protective coating 308 may comprise chitosan, and in other embodiments, the protecting coating 308 may comprise polyethylene glycol (PEG) and/or hydrogel films. Next, in FIG. 5E, the template 304 is removed, resulting in a nanomaterial coated 306 nanosensor 112. Exemplary images of an exemplary nanosensor 112 with a CNT/PAnNW coating are shown in FIG. 5F. According to some embodiments, the nanosensor 112 can include an IDE made of Au with 40 pairs of fingers. In some embodiments, the fingers can be of the following approximate dimensions: 3 mm×20 μm×70 nm (L×W×H)) and 20 μm gap on Mylar. With this protocol, nanosensors 112 can be manufactured with high reproducibility (see FIGS. 5A-5F). This protocol lays a basis for mass production of high-quality and uniform nanosensors 112.

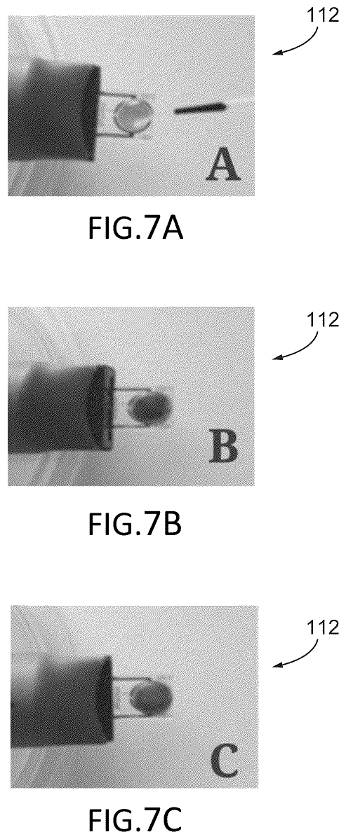

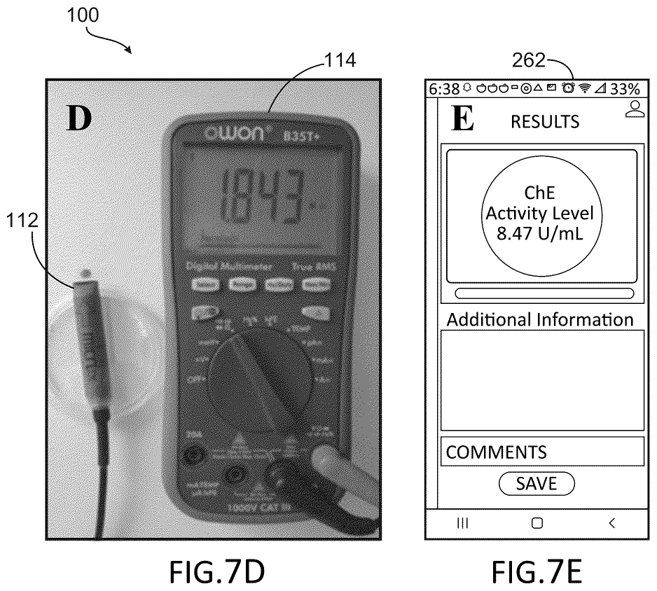

According to some embodiments, a pre-treatment pad 309 is placed atop the surface of the nanosensor 312 before a blood sample 500 is applied. After a ChE-specific pre-treatment period, a substrate 310 (e.g., reagent-preload glass fiber pad (3 mm diameter)) is placed on the top of the nanosensor 112 (FIG. 6) and then the nanosensor 112 may be connected via an adapter to a measurement component (e.g., a digital multimeter with wirelesss connectivity; see FIG. 7D) and data may be wirelessly transmitted to a processing device 120 (e.g., a smartphone) (FIG. 3A and FIGS. 4A-4B). The glass fiber pre-treatment pad 309 contains a selective AChE inhibitor and anti-buffer agents CaCl₂/MgCl₂ to minimize the effect of blood buffer system as the whole blood sample 500 containing AChE and BChE interacts with the nanosensor 112. After pretreatment, the substrate 310 loaded with acetylcholine (ACh) is placed onto the sensing surface and rapidly dissolves. According to an example embodiment, nanosensor system 100 can operate as follows as shown in FIG. 7: a pre-treatment pad 309 is placed atop the nanosensor film 306 coated with protective coating 308 (see FIG. 7A). Then, approximately 2 μL blood sample 500 is applied onto the nanosensor 112 (FIG. 7B). Substrate pad 310 is added to the nanosensor 112 (FIG. 7C) and data collection (e.g., via the measurement component 114) is initiated to record the data during enzyme reaction (FIG. 7D with reaction occurring) and the raw data and analytical results are displayed by processing device 120 (FIG. 7E). To achieve the best performance of the nanosensor, some parameters and conditions were first optimized with human AChE and BChE (Sigma), including the amount of CNT/PAnNWs, which determines the thickness of the nanocomposite on an IDE, the concentration of AChE substrates acetylcholine (ACh) (approximately 0.5 mM to 10 mM) pre-dried on a pad, the types of microporous pad for preloading (e.g. glass fiber or cellulose membrane), and the amount of protection film (e.g. chitosan, PEG, and/or hydrogels) on the top of nanosensor 112. Moreover, to minimize the blood matrix effect, improve sensitivity, and increase accuracy of the nanosensor, other protection films may be utilized.

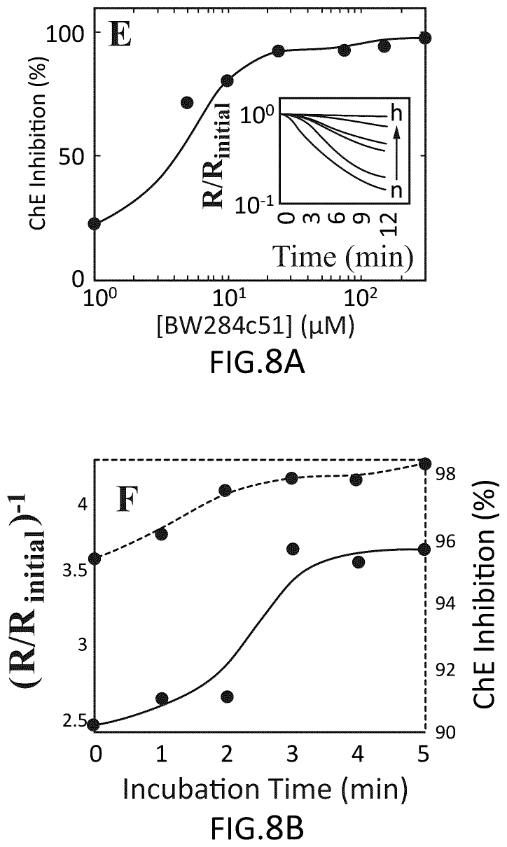

Normal human blood buffer systems (approximately at pH 7.4, bicarbonate buffer (major) and phosphate buffer (minor)) can neutralize protons generated by enzymatic reaction, thus affecting sensor performance (see FIG. 3E (b)). To address this issue, some cations Ca²⁺ and Mg²⁺ to minimize the effect of blood buffer systems on the sensor and specific AChE inhibitor BW 284c51 can be used to differentiate between AChE and BChE and were further optimized using blood samples by changing their amount in preloaded pads. In FIG. 8A, the concentration of BW284c51 inhibitor increases and inhibition of AChE activity also increases. Complete inhibition of AChE activity was achieved at 150 μM after using a 5 min incubation period. As such, 150 μM BW284c51 was selected for all future measurements of whole blood BChE activity, ensuring minimal interference from AChE. The incubation period for the pre-treatments was optimized and is provided in FIG. 8B. It was found that a 3 min incubation period was sufficient for the cations and released protons to effectively disrupt the carbonic acid/bicarbonate buffer capacity and for BW284c51 to inhibit all AChE activity within a whole blood sample. As such, 3 minutes was selected as the ideal incubation period for all pre-treatments of blood samples on the sensor surface. The protective film 308 was also optimized for reducing the blood matrix effect by adjusting its thickness on nanosensors 112. The nanosensor device may be prepared using these optimized conditions.

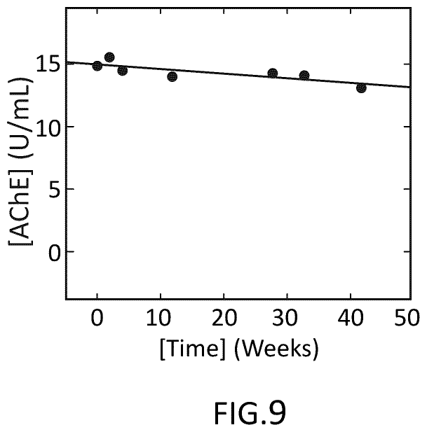

The reproducibility and stability of nanosensors under optimal conditions was tested by measuring enzyme activity with the same concentration of AChE in the time course of one year. The variation of measurements between nanosensors 112 was then statistically evaluated. In FIG. 9, results are shown from a long-term stability study of the nanosensor for determining AChE activity in whole blood over the course of one year, using a whole blood sample stored at −20 °C. A batch of nanosensors were prepared according to the present disclosure and stored in a vacuum desiccator at room temperature prior to testing. A baseline measurement (day 0) was established to determine if there is any loss in performance during subsequent testing. Nanosensors within the same batch exhibited exceptional stability for determining whole blood AChE activity over the course of one year. The RSD among all measurements within the batch was found to be 5.2 % and a decrease of 11.6% was determined between week 0 and week 42, indicating sufficient long-term stability. Without being beholden to any particular theory, the high stability may be attributed to the lack of biological recognition elements (e.g., enzymes or antibodies) and the inherently high stability of the MWCNT/PAnNF/CS nanocomposite film under dry conditions. While the nanosensor demonstrates excellent stability under dry conditions, exposure to high humidity levels can degrade the film over time due to gradual stripping of protons. As such, under controlled dry conditions, this platform demonstrates exceptional long-term stability for POC settings in comparison to other reported biosensors for determination of human exposure to pesticides.

The nanosensors 112 exhibit good uniformity, high reproducibility, and long-term stability with a coefficient of variation (CV) of less than 6% in one year.

In some instances, nanosensors 112 may include conducting polymer nanomaterials such as polypyrrole or polythiophene nanowires, or other new emerging conducting polymer nanomaterials.

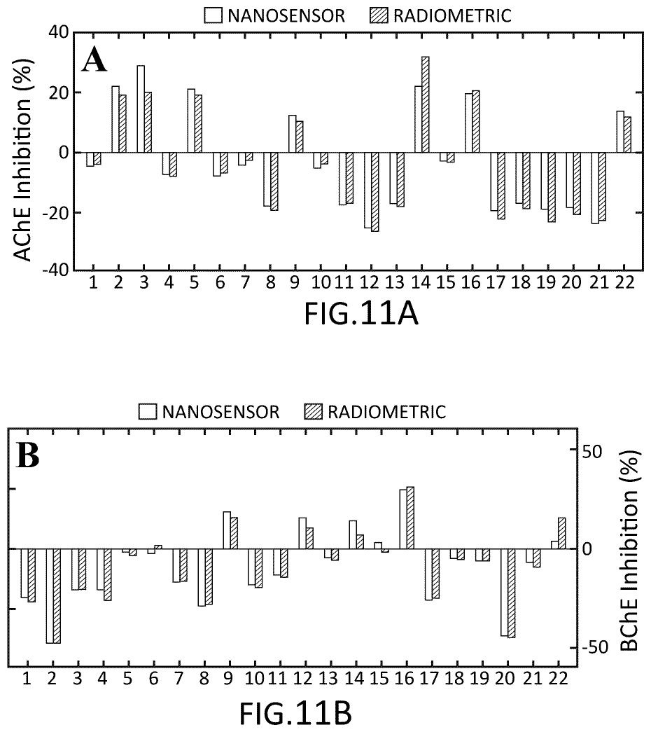

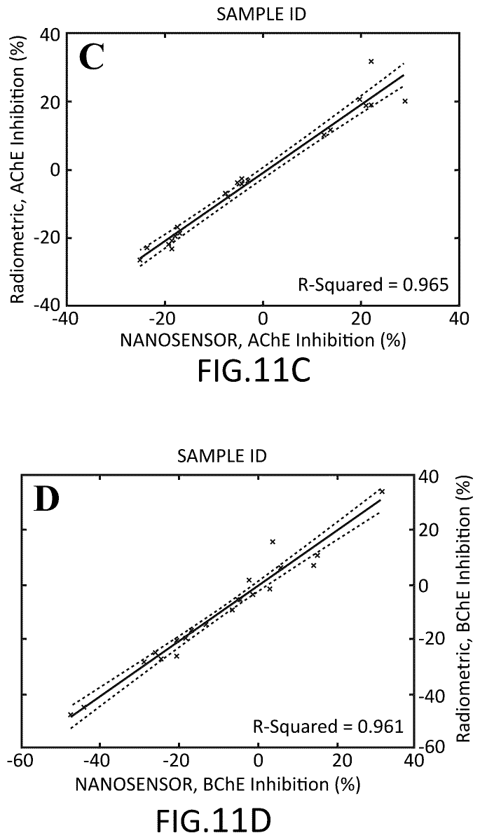

In order to achieve robust and unbiased results for the nanosensor system 100, its analytical metrics including the sensitivity, detection limit, dynamic range, and reproducibility are first defined. During the assay design and development, the quality of all biological and chemical reagents and samples from different vendors is ensured (see an attachment of authentication). Next, blank samples (controls), blind samples, standard samples (positive controls) are used for testing the device and making sure that the results from the nanosensor system 100 are accurate. Replicates and standard methods are used with quality-controlled equipment and procedures to ensure that the results from the nanosensor 100 are reproducible. Furthermore, inter-operator variability of nanosensors 112 is evaluated with multiple devices and samples. Finally, the results from the nanosensor system 100 are further validated with current standard methods, e.g., radiometric method (see FIG. 10A-11J and FIG. 11A-11D) .

A series of standard human erythrocyte AChE (H-AChE) and BChE (H-BChE) were spiked into heat denatured whole blood at concentrations (FIG. 3C and FIG. 3D) ranging from 0 to 18 U/mL H-AChE and 0-5 U/mL H-BChE, respectively. Under optimal conditions, 2 μL of a series of standard samples is sequentially applied to nanosensors 112. Accordingly, the conductance changes of nanosensors 112 is then recorded by the nanosensor system 100. The calibration curve of normalized resistance changes (R/Ri) vs. AChE or BChE concentrations is established. Six replicates of a blank sample and six AChE or BChE samples are analyzed by the nanosensor system 100. According to the calibration curves and replicate testing results, the analytical performance of the nanosensor system 112 is determined, including the dynamic range, sensitivity (the slope of the curve), and reproducibility. The detection limit of the nanosensor system 112 is determined based on 3 σ of low concentration/slope of the calibration curve. The calibration curve is used by the processing device 120 for blood testing.

In FIG. 10, four blood samples (each 20 mL) from healthy people (Zenbio, Inc) are aliquoted. One aliquot from each donor serves as a control (vehicle, without spiking with any pesticides) and the others from each donor are in vitro-spiked with different concentrations of a OP pesticide (Paraoxon-Methyl (PM) (0-820 ppb) or Diazinon-Oxon (DZO) (0-4.2 ppm)), and preincubated at 37 °C with different concentrations of each inhibitor for 1 hour to achieve enzyme inhibition ranging from 0 (vehicle), 5%, 10%, 20%, 30%, 50%, and 80% estimated by the Ellman assay method.

Under the optimal conditions, above vehicles and in vitro pre-incubated blood samples are tested with the nanosensor system 100 using 2 μL samples. Total blood ChE activity, plasma BChE activity, and blood AChE activity are measured for each sample by the following approach: A sensor measures total AChE activity with ACh. Then another sensor measures BChE activity with a BW AChE inhibitor included. To evaluate the reproducibility of the method, three replicates of a vehicle sample and a preincubated blood sample with a pesticide are analyzed by the nanosensor system 100.

In FIG. 10, under optimal nanosensor conditions, whole blood AChE and BChE for each sample were measured using 2.0 μL whole blood (4 μL total) in triplicates (total 300 testing). The reproducibility of the nanosensor for measuring in-vitro AChE and BChE spiked samples resulted in a CV of 2.75%. In parallel, whole blood AChE and BChE for the other half samples were analyzed using a standard radiometric method. The results of enzyme inhibition for each diazinon-oxon (FIG. 10A and C) or paraoxon-methyl (FIG. 10B and D) spiked whole blood sample from the two methods are summarized in FIG. 10. A linear regression model was built to initially understand the consistency between the nanosensor and the standard radiometric method. Briefly, AChE (FIG. 10E) and BChE (FIG. 10G) inhibition results from the two methods were plotted against each other, where each point represents sample inhibition measured by the nanosensor and radiometric method. High consistency between the nanosensor and radiometric measurements were observed, the linear correlation between the two for AChE (FIG. 10E, R²=0.974) and BChE (FIG. 10G, R²=0.961) are both greater than 0.95. Furthermore, the linear model equations for AChE and BChE were found to be y=0.9845x−0.5965 and y=0.9214x+0.5262, respectively. From these equations, it is clear that the two methods are consistent given that the slopes are close to 1 and the y-intercepts are close to 0. Next, the consistency between the two methods was further evaluated using Bland-Altman plots (FIGS. 10F and 10H). The mean value of the difference in inhibition for AChE and BChE, between the two methods, were −0.84% and −0.74% with 95% confidence intervals of (−7.7% to 6%) and (−8.4% to 6.9%), respectively. Linear model and Bland Altman plot for AChE activity between the nanosensor and Ellman's method are shown in FIG. 10I and FIG. 10J, respectively.

Approximately 25 migrant farmworkers in eastern North Carolina were recruited to work with the North Carolina Farmworkers Project. Briefly, a project field interviewer approached 3 to 4 farmworker labor camps in eastern North Carolina in the vicinity of Green, Harnett, Johnston, and Sampson Counties. The interviewer visited the camps in advance of the initial data collection, explained the project, and ensured that the resident farmworkers wanted to participate. At sample collection, the project participant requirements were again explained and 22 participants were recruited. A short interview was conducted and 1.5 drops of finger-stick blood samples (approximately 80 μL) were collected from each participant.

In FIG. 11, the enzyme activity obtained from the nanosensor system 100 and radiometric method for each farmworker for both times was determined. The enzyme inhibition (%) for each farmworker was calculated. The interview and cholinesterase data was entered into REDCap (Research Electronic Data Capture), a secure web-based system. Descriptive statistics were then generated to characterize the samples. The consistency between the two methods was statistically evaluated. Results from the nanosensor system 100 are in agreement with those from the radiometric method. These results demonstrated the feasibility of the nanosensor system 100 for onsite rapid, sensitive, and accurate detection of exposure to OP pesticides.

The results from nanosensor system 100 are consistent with the radiometric method (See FIGS. 11C and 11D). The nanosensor system 100 is highly sensitive with a detection limit (LOD) of 0.11 U/mL of AChE and 0.093 U/mL of BChE and reproducibility with CV of less than 5%, and it can detect low-level exposure (approximately 5 to 20% enzyme inhibition; see FIG. 11A and FIG. 11B). Based on normal human blood AChE and plasma BChE reference levels (approximately 5 to 15 U/mL and approximately 1 to 3.5 U/mL, respectively), the nanosensor system 100 is sensitive enough to achieve low level pesticide exposure.

The nanosensor system 100 achieved high sensitivity with the LOD of 0.11 U/mL AChE and 0.093 U/mL BChE, assay time of 10 minutes, sample volume (2 μL), and is able to detect low-level exposure (approximately 5 to 20% enzyme inhibition) in field.

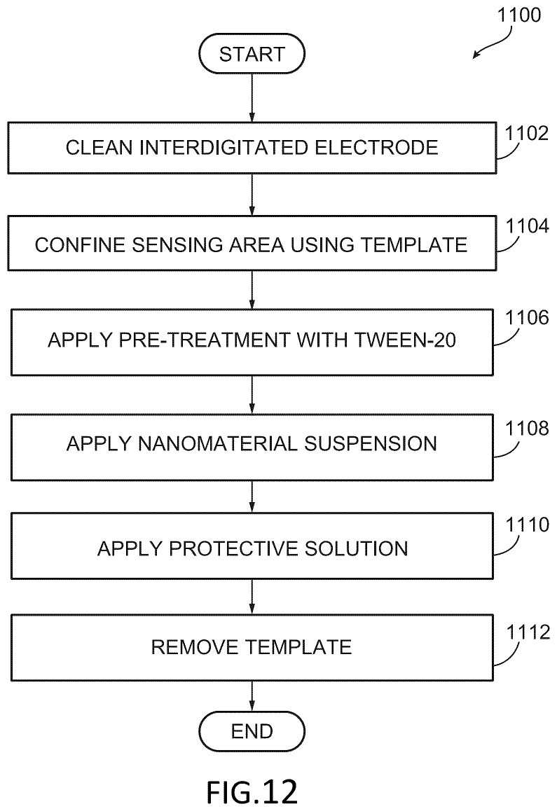

FIG. 12 is a flow diagram illustrating an exemplary method 1100 for constructing a nanosensor, according to exemplary embodiments consistent with the present disclosure. In block 1102, the method includes cleaning an interdigitated electrode 302 of nanosensor 112. In block 1104, the method includes confining the sensing area using a template 304. As discussed previously with respect to FIG. 6, the template 304 may include a hole with a diameter of approximately 3 mm. In block 1106, a pretreatment of the IDE with Tween-20 or other protective solution can be undertaken. In block 1108, the method can include applying a nanomaterial suspension comprising a nanomaterial coating 306 to the IDE 302. The nanomaterial coating 306 can comprise MWCNT/PAnNW in some embodiments. In other embodiments, the nanomaterial coating 306 can comprise polypyrrole or polythiophene nanowires, or other new emerging conducting polymer nanomaterials. In block 1110, the method can include applying a protective solution comprising a protective film 308. The protective film can comprise a chitosan film, a hydrogel, PEG, or combinations thereof. In block 1112, the method may include removing the template 304.

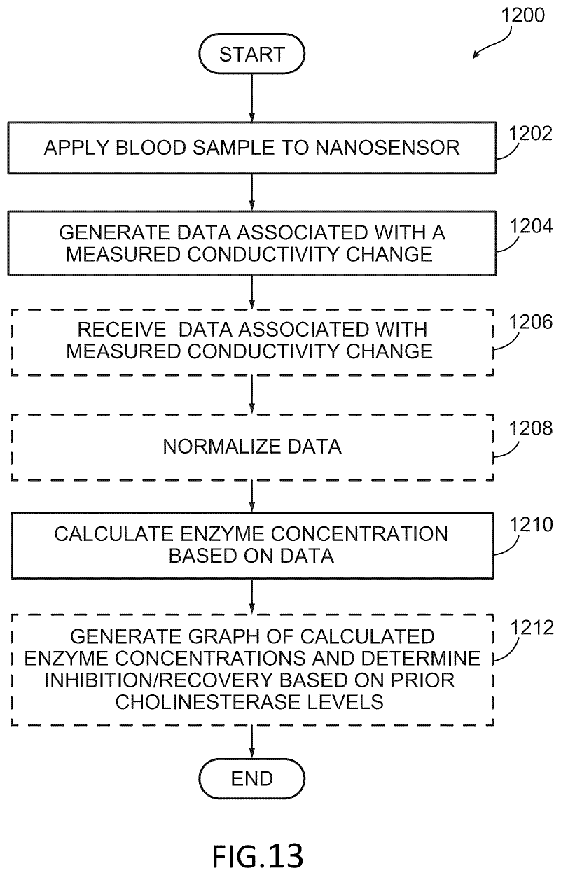

FIG. 13 is a flow diagram illustrating an exemplary method 1200 for assessing the exposure of a subject to a pesticide based on a blood sample, according to exemplary embodiments consistent with the present disclosure. In block 1202, the method can include applying a blood sample (e.g., blood sample 500) to a nanosensor 112. In block 1204, the method can include generating data associated with a conductivity change in nanosensor 112 in response to the addition of the blood sample 500. The data may be generated by a measurement component 114 (e.g., a multimeter) of nanosensor system 100. In optional block 1206, the method can include, receiving, by the processing device 120, data associated with the measured conductivity change. When nanosensor system 100 comprises a single device, the processing device 120 can be integrated with nanosensor platform 110, and accordingly, receiving may not be necessary. In optional block 1208, the method can include normalizing the data. According to some embodiments, normalization logic 224 of processing device 120 can utilize calibration data (e.g., calibration data 232) to calculate an enzyme concentration associated with the data received by the processing device 120 in response to the change in conductivity recorded by the measurement component 114. In block 1210, the method can include calculating enzyme concentration associated with the data based on a calibration curve. Based on the calculated enzyme concentration, the nanosensor system 100 can determine whether a subject from which the blood sample 500 was taken has been exposed to organophosphate pesticides. In optional block 1212, the processing device 120 of nanosensor system 100 can generate a graph depicting the calculated enzyme concentrations for a user of the nanosensor system 100 to view and evaluate the exposure of the subject to pesticides. In block 1212, inhibition and/or recovery can be determined based on prior cholinesterase levels.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In this description, numerous specific details have been set forth. It is to be understood, however, that implementations of the disclosed technology may be practiced without these specific details. In other instances, well-known methods, structures, and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrase “in one implementation” does not necessarily refer to the same implementation, although it may.

Throughout the specification and the claims, the following terms take at least the meanings explicitly associated herein, unless the context clearly dictates otherwise. The term “connected” means that one function, feature, structure, or characteristic is directly joined to or in communication with another function, feature, structure, or characteristic. The term “coupled” means that one function, feature, structure, or characteristic is directly or indirectly joined to or in communication with another function, feature, structure, or characteristic. The term “or” is intended to mean an inclusive “or.” Further, the terms “a,” “an,” and “the” are intended to mean one or more unless specified otherwise or clear from the context to be directed to a singular form. By “comprising” or “containing” or “including” is meant that at least the named element, or method step is present in article or method, but does not exclude the presence of other elements or method steps, even if the other such elements or method steps have the same function as what is named.

It is to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Although embodiments are described herein with respect to systems or methods, it is contemplated that embodiments with identical or substantially similar features may alternatively be implemented as systems, methods and/or non-transitory computer-readable media.

As used herein, unless otherwise specified, the use of the ordinal adjectives “first,” “second,” “third,” etc., to describe a common object, merely indicates that different instances of like objects are being referred to, and is not intended to imply that the objects so described must be in a given sequence, either temporally, spatially, in ranking, or in any other manner.

While certain embodiments of this disclosure have been described in connection with what is presently considered to be the most practical and various embodiments, it is to be understood that this disclosure is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the technology and also to enable any person skilled in the art to practice certain embodiments of this technology, including making and using any apparatuses or systems and performing any incorporated methods. The patentable scope of certain embodiments of the technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A nanosensor configured to detect pesticide analytes in a sample, the nanosensor comprising:
an interdigitated electrode (IDE); and
a nanomaterial deposited on the IDE, wherein the nanomaterial comprises a carbon nanoparticle phase and a polyaniline phase.

2. The nanosensor of claim 1, wherein the nanoparticle phase comprises carbon nanotubes (CNTs) and the polyaniline phase comprises polyaniline nanowires.

3. The nanosensor of claim 2, wherein the CNTs comprise between 0.5 and 1.5 wt %.

4. The nanosensor of claim 3, wherein the CNTs comprise 1.0 wt %.

5. The nanosensor of claim 1, further comprising a chitosan film deposited on the nanomaterial.

6. The nanosensor of claim 1, further comprising a substrate pad comprising a substrate.

7. The nanosensor of claim 6, further comprising a pre-treatment pad comprising one or more of a substrate inhibitor and one or more anti-buffer agents.

8. The nanosensor of claim 6, wherein the substrate is selected from acetylcholine, butyrylcholine, and combinations thereof.

9. The nanosensor of claim 7, wherein:
the substrate is selected from acetylcholine, butyrylcholine, and combinations thereof;
the substrate inhibitor is a selective acetylcholinesterase inhibitor; and
the one or more anti-buffer agents are selected from calcium chloride, magnesium chloride, and combinations thereof.

10. The nanosensor of claim 6, wherein the substrate pad has a microporous preloading structure.

11. The nanosensor of claim 10, wherein the microporous preloading structure is selected from glass fibers and cellulose membrane.

12. The nanosensor of claim 1, wherein the IDE is a gold IDE.

13. The nanosensor of claim 1, wherein the IDE comprises 40 pairs of fingers.

14. A system for detecting pesticide analytes in a sample, the system comprising:
a nanosensor configured to receive the sample;
a measurement component operatively coupled to the nanosensor configured to generate data associated with a measured conductivity change of the nanosensor in response to an addition of the sample to the nanosensor;
a processing device operatively coupled to the measurement component, the processing device comprising:
one or more processors; and
a non-transitory memory in communication with the one or more processors and storing instructions thereon that when executed by the one or more processors, are configured to cause the processing device to:
receive, from the measurement component, the data associated with the measured conductivity change; and
detect the presence of pesticide analytes based on the data.

15. The system of claim 14, wherein the non-transitory memory comprises further instructions that when executed by the one or more processors are configured to cause the processing device to calculate an enzyme concentration associated with the data based on a predetermined calibration curve.

16. The system of claim 14, wherein detecting the presence of pesticide analytes based on the data further comprises calculating an enzyme concentration based on the data.

17. The system of claim 16, wherein the enzyme is selected from acetylcholinesterase, butyrylcholinesterase, and combinations thereof.

18. The system of claim 14, wherein the pesticide analytes are organophosphate pesticides.

19. The system of claim 14, wherein the sample is selected from a blood sample, a food sample, and a water sample.

20. The system of claim 19, wherein the sample is an environmental water sample.

21. The system of claim 14, wherein the nanosensor comprises a nanomaterial modified interdigitated electrode (IDE).

22. The system of claim 21, wherein the nanomaterial comprises carbon nanotube polyaniline nanowires.

23. The system of claim 14, wherein the measurement component comprises a multimeter.

24. The system of claim 14, wherein the processing device is operatively coupled to the measurement component through a wireless communication protocol.

25. A method comprising:
applying a sample to a nanosensor;
generating, by a measurement component operatively coupled to the nanosensor, data associated with a measured conductivity change of the nanosensor in response to applying the sample to the nanosensor;
receiving, by a processing device operatively coupled to the measurement component, the data associated with the measured conductivity change; and
determining, by the processing device, whether the sample contains pesticide analytes based on the data.

26. The method of claim 25 further comprising calculating an enzyme concentration, by the processing device, associated with the data based on a predetermined calibration curve.

27. The method of claim 25, wherein determining whether the sample contains pesticide analytes based on the data further comprises calculating an enzyme concentration based on the data by the processing device.

28. The method of claim 27, wherein the enzyme is selected from acetylcholinesterase, butyrylcholinesterase, and combinations thereof.

29. The method of claim 25, wherein the nanosensor comprises a nanomaterial modified interdigitated electrode (IDE).

30. The method of claim 29, wherein the nanomaterial comprises carbon nanotube polyaniline nanowires.

31. The method of claim 25, wherein the measurement component comprises a multimeter.

32. The method of claim 25, wherein the data is received by the processing device and from the measurement component by a wireless communication protocol.

33. The method of claim 25, wherein the pesticide analytes are organophosphate pesticides.

34. The method of claim 25, wherein the sample is selected from a blood sample, a food sample, and a water sample.

35. The method of claim 34, wherein the sample is an environmental water sample.
